(19) United States
(12) Patent Application Publication
(10) Pub. No.: US 2024/0101502 A1
(43) Pub. Date: Mar. 28, 2024

(54) METHOD FOR PRODUCING (METH)ACROLEIN AND/OR (METH)ACRYLIC ACID

(71) Applicant: Mitsubishi Chemical Corporation, Tokyo (JP)

(72) Inventor: Keisuke Baba, Tokyo (JP)

(73) Assignee: Mitsubishi Chemical Corporation, Tokyo (JP)

(21) Appl. No.: 18/516,423

(22) Filed: Nov. 21, 2023

Related U.S. Application Data
(63) Continuation of application No. PCT/JP2022/021177, filed on May 24, 2022.

(30) Foreign Application Priority Data
May 25, 2021 (JP) 2021-087587

(51) Int. Cl. C07C 45/28

(52) U.S. Cl. CPC C07C 45/28

(57) ABSTRACT

A production method of one or both of (meth)acrolein and (meth)acrylic acid using a heat-exchange-type reaction vessel having a reaction tube at an inner part is provided, the method including causing an oxidation reaction of a raw material supplied to the reaction tube while circulating a heat medium to an outer side of the reaction tube to produce one or both of (meth)acrolein and (meth)acrylic acid, in which the reaction tube has i layers, which are a plurality of catalyst layers having different catalyst charging amounts per unit volume, in a longitudinal direction of the reaction tube, provided that i is an integer of 2 or more, and the oxidation reaction satisfies Expression (1).

ξ≤0.275(mol·K·h⁻¹·W⁻¹) (1)

Provided that (AAA) is satisfied.

ξ=F×(m1/Σⱼ₌₁ⁱmj)/(U×A)^{. . . (*)} (AAA)

### CROSS-REFERENCES TO RELATED APPLICATIONS

This application is a continuation of International Application No. PCT/JP2022/021177 filed on May 24, 2022, which claims priority to and the benefit of Japanese Patent Application No. 2021-087587, filed May 25, 2021, the content of which is incorporated herein by reference.

### BACKGROUND OF THE INVENTION

### Field of the Invention

The present invention relates to a production method of one or both of (meth)acrolein and (meth)acrylic acid.

### Background Art

In general, large reaction heat is generated when producing (meth)acrolein or (meth)acrylic acid by an oxidation reaction, and thus a heat-exchange-type reaction vessel in which a reaction is carried out by circulating a heat medium to an outer side of a reaction tube is being widely used. However, because the flow of the heat medium is not uniform in the reaction vessel, a large deviation is generated in the heat medium temperature in the reaction vessel. In the inner part of the reaction tube present in a region in which the temperature of the heat medium is high, a by-product is generated because of the excessive progression of a reaction, or a local catalyst degradation progresses, which lowers the selectivity of a target product. In addition, reaction heat that exceeds the heat removal capacity of the reaction vessel is generated, which may cause a runaway reaction.

As a measure for such a deviation in the heat medium temperature in the heat-exchange-type reaction vessel, for example, JP 2010-132584 A (Patent Document 1) discloses a method in which, in a gas-phase oxidation reaction using a multi-tubular heat-exchange-type reaction vessel having at least a plurality of reaction tubes having a catalyst layer to which a catalyst has been charged, the conditions are set for a raw material gas flow rate and for a heat removal capacity of the reaction vessel in consideration of the heat quantity balance of a heat medium.

### SUMMARY OF INVENTION

### Technical Problem

However, when the conditions are set by merely focusing on the heat quantity balance of the heat medium, there is a probability of not achieving a target production amount because a required amount of a catalyst cannot be charged to the reaction tube, or shortening of a continuous operation period of the reaction vessel. Alternatively, there is a probability of an increase in manufacturing costs because the reaction vessel is equipment that is excessively large for the production amount of a target product.

Therefore, an object of the present invention is to produce one or both of (meth)acrolein and (meth)acrylic acid with a high selectivity by preventing the runaway reaction in a heat-exchange-type reaction vessel and preventing the excessive progression of a reaction and a local catalyst degradation. The term “(meth)acrolein” is a generic term for acrolein and methacrolein, and the term “(meth)acrylic acid” is a generic term for acrylic acid and methacrylic acid.

### Solution to Problem

[1]: A production method of one or both of (meth)acrolein and (meth)acrylic acid using a heat-exchange-type reaction vessel comprising a reaction tube at an inner part, the production method comprising causing an oxidation reaction of a raw material supplied to the reaction tube while circulating a heat medium to an outer side of the reaction tube to produce one or both of (meth)acrolein and (meth)acrylic acid,
wherein the reaction tube comprises i layers, which are a plurality of catalyst layers having different catalyst charging amounts per unit volume, in a longitudinal direction of the reaction tube, provided that i is an integer of 2 or more, and
the oxidation reaction satisfies Expression (1).

ξ≤0.275(mol·K/h/W) (1)

Provided that the following formula is satisfied.

ξ=F×(m1/Σⱼ₌₁ⁱmj)/(U×A) (*)

In Formula (*), m1 is a catalyst charging amount (kg) in a first catalyst layer from a raw material inlet side of the reaction tube; mj is a catalyst charging amount (kg) in a j-th catalyst layer from the raw material inlet side of the reaction tube; j is an integer of 1 or more and i or less; F is a supply amount (mol/h) of the raw material supplied to the reaction tube; A is an inner surface area (m²) of the reaction tube with which the first catalyst layer from the raw material inlet side of the reaction tube comes into contact; and U is an overall heat transfer coefficient (W/m²/K) based on an inner surface area of a portion in the reaction tube with which both the first catalyst layer and the heat medium come into contact.

[2]: The production method according to [1], wherein the oxidation reaction further satisfies Expression (1′).

0.002≤ξ≤0.275(mol·K/h/W) (1′)

[3]: The production method according to [1] or [2], wherein the oxidation reaction further satisfies Expression (1″).

ξ≤0.24(mol·K/h/W) (1″)

[4]: The production method according to any one of [1] to [3], wherein the oxidation reaction further satisfies Expression (2).

0.25≤τ≤0.5 (2)

Provided that the following formula is satisfied.

$$τ = m 1 / {\sum }_{k = 1}^{i} mk \qquad \text{(*} \text{*)}$$

In Formula (**), m1 is the catalyst charging amount (kg) in the first catalyst layer from the raw material inlet side of the reaction tube; mk is a catalyst charging amount (kg) in a k-th catalyst layer from the raw material inlet side of the reaction tube; and k is an integer of 1 or more and i or less.

[5]: The production method according to [4], wherein the oxidation reaction further satisfies Expression (2′).

0.26≤τ≤0.5 (2′)

[6]: The production method according to any one of [1] to [5], wherein i as the number of the catalyst layers of the reaction tube is 2 to 4.

[7]: The production method according to any one of [1] to [6], wherein U is 40 to 400 (W/m²/K).

[8]: The production method according to any one of [1] to [7], wherein U is 50 to 300 (W/m²/K).

[9]: The production method according to any one of [1] to [8], wherein F is 1 to 20 (mol/h).

[10]: The production method according to any one of [1] to [9], wherein F is 2.5 to 15 (mol/h).

[11]: The production method according to any one of [1] to [10], wherein A is 0.03 to 0.6 (m²).

[12]: The production method according to any one of [1] to [11], wherein the raw material is at least one selected from propylene, isobutylene, tert-butanol, and methyl tert-butyl ether, and one or both of the (meth)acrolein and the (meth)acrylic acid are (meth)acrolein and (meth)acrylic acid.

[13]: The production method according to any one of [1] to [11], wherein the raw material is (meth)acrolein, and one or both of the (meth)acrolein and the (meth)acrylic acid are (meth)acrylic acid.

[14]: The production method according to [12], wherein a catalyst having a formulation represented by Formula (I) is used in the catalyst layer.

Moₐ₁Bi_{b1}Fe_{c1}M_{d1}Xₑ₁Y_{f1}Z_{g1}Siₕ₁Oᵢ₁ (I)

In Formula (I), Mo, Bi, Fe, Si, and O each represent molybdenum, bismuth, iron, silicon, and oxygen. M represents at least one element selected from the group consisting of cobalt and nickel. X represents at least one element selected from the group consisting of chromium, lead, manganese, calcium, magnesium, niobium, silver, barium, tin, tantalum, and zinc. Y represents at least one element selected from the group consisting of phosphorus, boron, sulfur, selenium, tellurium, cerium, tungsten, antimony, and titanium. Z represents at least one element selected from the group consisting of lithium, sodium, potassium, rubidium, cesium, and thallium. a1, b1, c1, d1, e1, f1, g1, h1, and i1 represent an atomic ratio of each of the elements, provided that when a1=12, b1=0.01 to 3, c1=0.01 to 5, d1=1 to 12, e1=0 to 8, f1=0 to 5, g1=0.001 to 2, h1=0 to 20, and i1 is an atomic ratio of oxygen required to satisfy a valence of the element.

[15] The production method according to [13], wherein a catalyst having a formulation represented by Formula (II) is used in the catalyst layer.

Pₐ₂Mo_{b2}V_{c2}Cu_{d2}Xₑ₂Y_{f2}Z_{g2}Oₕ₂ (II)

In Formula (II), P, Mo, V, Cu, and O each represent phosphorus, molybdenum, vanadium, copper, and oxygen. X represents at least one element selected from the group consisting of antimony, bismuth, arsenic, germanium, zirconium, tellurium, silver, selenium, silicon, tungsten, and boron. Y represents at least one element selected from the group consisting of iron, zinc, chromium, magnesium, tantalum, cobalt, manganese, barium, gallium, cerium, and lanthanum. Z represents at least one element selected from the group consisting of potassium, rubidium, cesium, and thallium. a2, b2, c2, d2, e2, f2, g2, and h2 represent an atomic ratio of each of the elements, provided that when b2=12, a2=0.5 to 3, c2=0.01 to 3, d2=0.01 to 2, e2=0 to 3, f2=0 to 3, g2=0.01 to 3, and h2 is an atomic ratio of oxygen required to satisfy a valence of the element.

### Advantageous Effects of Invention

According to the production method of the present invention, one or both of (meth)acrolein and (meth)acrylic acid can be produced with a high selectivity by preventing the runaway reaction in a heat-exchange-type reaction vessel and preventing the excessive progression of a reaction and a local catalyst degradation.

### BRIEF DESCRIPTION OF DRAWINGS

FIG. 1 is a schematic vertical cross-sectional view showing an example of a heat-exchange-type reaction vessel used in the present invention.

### DETAILED DESCRIPTION OF THE INVENTION

Hereinafter, the embodiment of the present invention will be described in detail.

In the present specification, a numerical value range represented using “to” means a range including the numerical values written before and after “to” as a lower limit value and an upper limit value, and “A to B” means A or more and B or less.

The dimension of the drawing and the like described by an example in the following description are examples, and the present invention is not necessarily limited thereto and can be implemented with appropriate changes in a range not changing the scope thereof.

A production method of one or both of (meth)acrolein and (meth)acrylic acid of the present invention (hereinafter, “production method of one or both of (meth)acrolein and (meth)acrylic acid” may also be simply referred to as “production method”) using a heat-exchange-type reaction vessel having a reaction tube at an inner part is a method of causing an oxidation reaction of a raw material supplied to the reaction tube while circulating a heat medium to an outer side of the reaction tube, in which the reaction tube has i layers, which are a plurality of catalyst layers having different catalyst charging amounts per unit volume, in a longitudinal direction of the reaction tube. Provided that i is an integer of 2 or more. Furthermore, in the production method of the present invention, the oxidation reaction satisfies Expression (1).

ξ≤0.275(mol·K/h/W) (1)

Provided that the following formula is satisfied.

ξ=F×(m1/Σⱼ₌₁ⁱmj)/(U×A) (*)

In Formula (*), m1 is a catalyst charging amount (kg) in a first catalyst layer from a raw material inlet side of the reaction tube; mj is a catalyst charging amount (kg) in an j-th catalyst layer from the raw material inlet side of the reaction tube; j is an integer of 1 or more and i or less; F is a supply amount (mol/h) of the raw material supplied to the reaction tube; A is an inner surface area (m²) of the reaction tube with which the first catalyst layer from the raw material inlet side of the reaction tube comes into contact; and U is an overall heat transfer coefficient (W/m²/K) based on an inner surface area of a portion in the reaction tube with which both the first catalyst layer and the heat medium come into contact.

When the production method of the present invention satisfies the above-mentioned conditions, the runaway reaction in the heat-exchange-type reaction vessel is prevented, and also the excessive progression of a reaction and a local catalyst degradation are prevented, thereby making it possible to produce one or both of (meth)acrolein and (meth)acrylic acid with a high selectivity.

The reason therefor is presumed as follows.

In the production of one or both of (meth)acrolein and (meth)acrylic acid using the heat-exchange-type reaction vessel, as a reaction temperature increases, a heat generation rate in the reaction vessel increases exponentially, but a heat removal rate increases only linearly. Therefore, the heat removal capacity of the reaction vessel becomes insufficient as the reaction progresses, and an increase in the reaction temperature causes excessive progression of a reaction and a local catalyst degradation. Furthermore, when the heat removal capacity of the reaction vessel becomes excessively insufficient, a runaway reaction may occur.

On the other hand, by appropriately distributing a catalyst to each of the catalyst layers using the reaction tube having a plurality of the catalyst layers having different catalyst charging amounts, the ratio between the heat generation rate and the heat removal rate of each of the catalyst layers of the reaction vessel can be controlled while ensuring a required catalyst charging amount. The present invention particularly focuses on the first catalyst layer having a highest raw material concentration to control the ratio between the heat generation rate and the heat removal rate in the above-mentioned catalyst layer. Then, when the ratio between the heat generation rate and the heat removal rate in the first catalyst layer is set to a value suitable for producing one or both of (meth)acrolein and (meth)acrylic acid, the ratio between the heat generation rate and the heat removal rate of each of the catalyst layers of the reaction tube becomes appropriate, which makes it possible to minimize an increase in the reaction temperature. This prevents a runaway reaction, thereby making it possible to produce one or both of (meth)acrolein and (meth)acrylic acid with a high selectivity.

[Production Method]

The production method of the present invention can be suitably applied to a production method of (meth)acrolein and (meth)acrylic acid using, as a raw material, at least one selected from propylene, isobutylene, tert-butanol, and methyl tert-butyl ether, or to a production method of (meth)acrylic acid using (meth)acrolein as a raw material. In particular, the application to a production method of (meth)acrylic acid using (meth)acrolein as a raw material is preferable, and the application to a production method of methacrylic acid from methacrolein is more preferable.

<Heat-Exchange-Type Reaction Vessel>

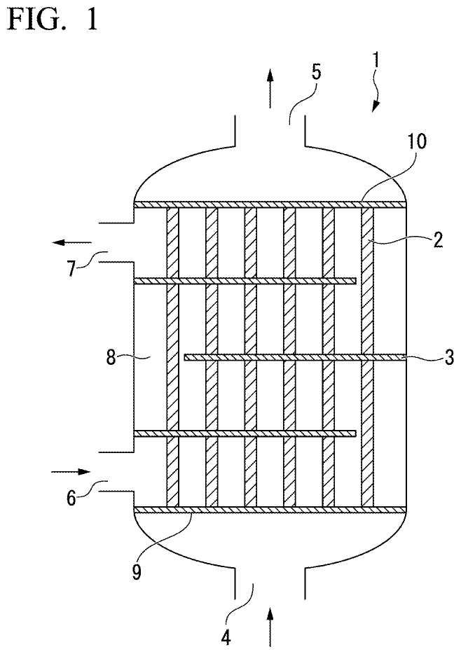

In the production method of the present invention, the heat-exchange-type reaction vessel having the reaction tube at the inner part is used. As the heat-exchange-type reaction vessel, for example, a double-tube heat-exchange-type reaction vessel and, industrially, a multi-tubular heat-exchange-type reaction vessel can be used. FIG. 1 shows an example of the multi-tubular heat-exchange-type reaction vessel.

In FIG. 1, a reaction vessel 1 has a reaction tube 2 and a baffle plate 3 at the inner part. A raw material inlet part 4 is provided at the lower part of the reaction vessel 1, and a product outlet part 5 is provided at the upper part thereof. The inner part of the reaction vessel 1 is divided into three regions in an up and down direction by a first partition plate 9 on the raw material inlet part 4 side and a second partition plate 10 on the product outlet part 5 side. Each of the reaction tubes 2 is provided to extend from the first partition plate 9 to the second partition plate 10, and each of both edge surfaces of the reaction tube 2 is open to the raw material inlet part 4 side and the product outlet part 5 side. In the region between the first partition plate 9 and the second partition plate 10 in the reaction vessel 1, a heat medium bath 8 that circulates a heat medium is provided on the outer side of the reaction tube 2. A heat medium inlet part 6 is provided to be close to the first partition plate 9 on the side wall of the reaction vessel 1, and a heat medium outlet part 7 is provided to be close to the second partition plate 10 thereon. In the region between the first partition plate 9 and the second partition plate 10, the baffle plate 3 is provided to be orthogonal to the longitudinal direction of the reaction tube 2. In the reaction vessel 1, a raw material flows in from the raw material inlet part 4, flows through the reaction tube 2 from the raw material inlet side of the reaction tube 2, and flows out from the product outlet part 5. The raw material inlet side of the reaction tube 2 means the edge surface on the raw material inlet part 4 side. A heat medium flows in from the heat medium inlet part 6, flows through the outer side of the reaction tube 2 from the raw material inlet part 4 side to the product outlet part 5 side in a meandering manner by the baffle plate 3, and flows out from the heat medium outlet part 7.

<Catalyst Layer of Reaction Tube>

In the reaction vessel used in the production method of the present invention, the reaction tube has i layers, which are a plurality of the catalyst layers having different catalyst charging amounts per unit volume, in the longitudinal direction of the reaction tube. Provided that i is an integer of 2 or more. This means that the reaction tube has two or more catalyst layers in the longitudinal direction thereof, and that adjacent catalyst layers have different catalyst charging amounts from each other per unit volume. In addition, the phrase “different catalyst charging amounts per unit volume” means that the catalyst charging amounts per unit volume are different by 1% or more. When the catalyst layer contains a diluent material to be described later, this includes a case in which the mixing ratio of the charged catalyst and the diluent material is different by 1% or more, or a case in which the density of the catalyst or the diluent material is different by 1% or more. When the reaction tube has a thickness of 100 mm or more in the longitudinal direction, this is regarded as the “catalyst layer”.

In order to form the catalyst layers having different catalyst charging amounts per unit volume, for example, there is a method of charging by mixing a diluent material and a catalyst such that the catalyst charging amount per unit volume is a desired value, or a method of charging catalysts having different shapes. From the viewpoint of catalyst manufacturing costs, a method of charging by mixing a diluent material and a catalyst is preferable.

The diluent material is not particularly limited as long as it is an inert substance that is not active in the oxidation reaction for producing one or both of (meth)acrolein and (meth)acrylic acid. Examples of the inert substances include silica, alumina, silica-alumina, silicon carbide, titania, magnesia, ceramic balls, and stainless steel.

i as the number of the catalyst layers is not particularly limited as long as it is an integer of 2 or more. From the viewpoint of reducing the load of a catalyst charging operation, i is preferably an integer of 4 or less, and is more preferably 2. When i is 2, the catalyst charging amount per unit volume in the first catalyst layer from the raw material inlet side of the reaction tube is preferably smaller than the catalyst charging amount per unit volume in the second catalyst layer from the viewpoint of controlling the reaction heat on the raw material inlet side of the reaction tube.

The length of the catalyst layer is not particularly limited, but from the viewpoint of the production amount of one or both of (meth)acrolein and (meth)acrylic acid, the length of the first catalyst layer from the raw material inlet part of the reaction tube is preferably 0.5 m or more, and more preferably 1.5 m or more. In addition, from the viewpoint of manufacturing costs, the length of the first catalyst layer from the raw material inlet part of the reaction tube is preferably 6 m or less, and more preferably 4 m or less.

In addition, between the end part on the raw material inlet side and the catalyst layer, the reaction tube may have an inert substance layer for purposes such as supporting the catalyst layer and preheating a raw material. The inert substance layer is preferably formed from only the above-mentioned inert substance. In case of using a method of charging by mixing a diluent material and a catalyst when forming the catalyst layer, this is preferable because, by using the same inert substance as the diluent material used in the catalyst layer, sampling and sieving operations after the completion of the oxidation reaction become simple.

<Catalyst>

It is preferable that catalysts charged to each of the catalyst layers be composed of common elements, and that a difference in a compositional ratio of each of the elemental components be 10% or less.

When the production method of the present invention is a production method of (meth)acrolein and (meth)acrylic acid using, as a raw material, one or more selected from propylene, isobutylene, tert-butanol, and methyl tert-butyl ether, it is preferable to use a catalyst having a formulation represented by Formula (I) in the above-mentioned catalyst layer.

Moₐ₁Bi_{b1}Fe_{c1}M_{d1}Xₑ₁Y_{f1}Z_{g1}Siₕ₁Oᵢ₁ (I)

In Formula (I), Mo, Bi, Fe, Si, and O each represent molybdenum, bismuth, iron, silicon, and oxygen. M represents at least one element selected from the group consisting of cobalt and nickel. X represents at least one element selected from the group consisting of chromium, lead, manganese, calcium, magnesium, niobium, silver, barium, tin, tantalum, and zinc. Y represents at least one element selected from the group consisting of phosphorus, boron, sulfur, selenium, tellurium, cerium, tungsten, antimony, and titanium. Z represents at least one element selected from the group consisting of lithium, sodium, potassium, rubidium, cesium, and thallium. a1, b1, c1, d1, e1, f1, g1, h1, and i1 represent an atomic ratio of each of the elements, provided that when a1=12, b1=0.01 to 3, c1=0.01 to 5, d1=1 to 12, e1=0 to 8, f1=0 to 5, g1=0.001 to 2, h1=0 to 20, and i1 is an atomic ratio of oxygen required to satisfy a valence of each of the above-mentioned elements.

In addition, when the production method of the present invention is a production method of (meth)acrylic acid using (meth)acrolein as a raw material, it is preferable to use a catalyst having a formulation represented by Formula (II) in the above-mentioned catalyst layer.

Pₐ₂Mo_{b2}V_{c2}Cu_{d2}Xₑ₂Y_{f2}Z_{g2}Oₕ₂ (II)

In Formula (II), P, Mo, V, Cu, and O each represent phosphorus, molybdenum, vanadium, copper, and oxygen. X represents at least one element selected from the group consisting of antimony, bismuth, arsenic, germanium, zirconium, tellurium, silver, selenium, silicon, tungsten, and boron. Y represents at least one element selected from the group consisting of iron, zinc, chromium, magnesium, tantalum, cobalt, manganese, barium, gallium, cerium, and lanthanum. Z represents at least one element selected from the group consisting of potassium, rubidium, cesium, and thallium. a2, b2, c2, d2, e2, f2, g2, and h2 represent an atomic ratio of each of the elements, provided that when b2=12, a2=0.5 to 3, c2=0.01 to 3, d2=0.01 to 2, e2=0 to 3, f2=0 to 3, g2=0.01 to 3, and h2 is an atomic ratio of oxygen required to satisfy a valence of each of the above-mentioned elements.

The shape, the size, and the like of the catalyst are not particularly limited, and it is possible to use a catalyst which has a spherical shape, a column shape, a ring shape, a star shape, or the like and molded using a general tablet molding machine, a general extrusion molding machine, a general granulating machine, or the like. In addition, a supported catalyst in which the catalyst having the above-mentioned formulation is supported on a carrier may be used.

For the catalyst, catalysts having a plurality of shapes may be used, but the same shape is preferable from the viewpoint of the manufacturing cost of the catalyst.

<Oxidation Reaction>

In the present invention, the production method of one or both of (meth)acrolein and (meth)acrylic acid by an oxidation reaction of a raw material will be described using FIG. 1. A raw material flows in from the raw material inlet part 4 and is supplied from the raw material inlet side of the reaction tube 2. Then, the raw material is brought into contact with the catalyst layer of the reaction tube 2 while removing reaction heat by allowing the heat medium bath 8 provided on the outer side of the reaction tube 2 to circulate a heat medium, thereby making it possible to produce one or both of (meth)acrolein and (meth)acrylic acid.

In the production method of one or both of (meth)acrolein and (meth)acrylic acid of the present invention, the oxidation reaction of the raw material satisfies Expression (1). In Expression (1), the value of ξ represents the easiness of temperature change in the first catalyst layer from the raw material inlet side of the reaction tube. When ξ satisfies Expression (1), the amount of the catalyst distributed to the first catalyst layer is appropriate, which makes the ratio between the heat generation rate and the heat removal rate suitable for producing one or both of (meth)acrolein and (meth)acrylic acid. In Expression (1), the upper limit of the value of ξ is preferably 0.24 (mol·K/h/W) or less, more preferably 0.12 (mol·K/h/W) or less, and further preferably 0.06 (mol·K/h/W) or less. In addition, the lower limit of the value of ξ is preferably 0.002 (mol·K/h/W) or more. Thus, the manufacturing cost with respect to the production amount of one or both of (meth)acrolein and (meth)acrylic acid can be reduced. The lower limit of the value of ξ is more preferably 0.004 (mol·K/h/W) or more, and further preferably 0.006 (mol·K/h/W) or more.

When the heat-exchange-type reaction vessel has a plurality of reaction tubes, it is preferable that 50% or more of the total number of the above-mentioned plurality of reaction tubes satisfy Expression (1), it is more preferable that 80% or more of the total number of the above-mentioned plurality of reaction tubes satisfy Expression (1), and it is further preferable that 90% or more of the total number of the above-mentioned plurality of reaction tubes satisfy Expression (1).

The ratio i of the catalyst charging amount of the first catalyst layer from the raw material inlet side of the above-mentioned reaction tube to the total catalyst charging amount of the reaction tube is represented by Formula (**).

$$τ = m 1 / {\sum }_{k = 1}^{i} mk \qquad \text{(*} \text{*)}$$

In Formula (**) above, m1 is the catalyst charging amount (kg) in the first catalyst layer from the raw material inlet side of the reaction tube; mk is the catalyst charging amount (kg) in a k-th catalyst layer from the raw material inlet side of the reaction tube; and k is an integer of 1 or more and i or less.

i is not particularly limited as long as ξ=F×τ/(U×A) satisfies Expression (1), but it is preferable that Expression (2) be satisfied.

0.25≤τ≤0.5 (2)

When the value of τ is 0.25 or more, the amount of heat generated in catalyst layers after the second layer from the raw material inlet side of the reaction tube is more suitably reduced. In addition, when the value of τ is 0.5 or less, the productivity of one or both of (meth)acrolein and (meth)acrylic acid is improved. The lower limit of the value of τ is more preferably 0.26 or more, further preferably 0.27 or more, and particularly preferably 0.28 or more. In addition, the lower limit of the value of τ is more preferably 0.48 or less, further preferably 0.46 or less, and particularly preferably 0.45 or less.

When the heat-exchange-type reaction vessel has a plurality of reaction tubes, it is preferable that 50% or more of the total number of the above-mentioned plurality of reaction tubes satisfy Expression (2), it is more preferable that 80% or more of the total number of the above-mentioned plurality of reaction tubes satisfy Expression (2), and it is further preferable that 90% or more of the total number of the above-mentioned plurality of reaction tubes satisfy Expression (2).

U in Formula (*) is an overall heat transfer coefficient (W/m²/K) based on the inner surface area of the portion in the above-mentioned reaction tube with which both the above-mentioned first catalyst layer and the above-mentioned heat medium come into contact. As a method of calculating U, an inert gas composed of 21% by volume of oxygen and 79% by volume of nitrogen is supplied to the reaction tube at a temperature 50° C. lower than that of the heat medium circulating the outer side of the reaction tube, and calculation can be performed from the results of measuring the temperature distribution in a minute region of the first catalyst layer (hereinafter, “minute region of the first catalyst layer” may be simply referred to as “minute region”). The minute region indicates a region obtained by dividing the reaction tube into 20 mm in the longitudinal direction. A temperature change dT1 (K) in the minute region in the longitudinal direction of the reaction tube is obtained by Formula (***) when a difference between the temperature of the heat medium circulating the outer side of the reaction tube and the average temperature in the minute region is defined as dT2 (K).

dT1=U×inner surface area(m²) of reaction tube with which minute region is in contact with×dT2/[mass flow rate(g/s) of inert gas×specific heat(J/g/K) of inert gas] (***)

U can be calculated by obtaining a value of U at which dT1 calculated by Formula (***) and the actual measurement value of dT1 coincide with each other using a least squares method. U is an arithmetic mean value obtained for 20 adjacent minute regions. In addition, at least one minute region of the 20 minute regions is set such that dT2 is 3° C. or higher. When obtaining U, the same conditions as those for the above-mentioned oxidation reaction are used except that, instead of a raw material, the above-mentioned inert gas is supplied at a temperature 50° C. lower than that of the heat medium circulating the outer side of the reaction tube.

U is not particularly limited as long as satisfies Expression (1), but from the viewpoint of reducing the amount of heat generated in the catalyst layer, U is preferably 40 (W/m²/K) or more, more preferably 60 (W/m²/K) or more, and further preferably 70 (W/m²/K) or more. In addition, from the economical viewpoint of preventing the reaction vessel from becoming expensive, U is preferably 400 (W/m²/K) or less, more preferably 300 (W/m²/K) or less, and further preferably 150 (W/m²/K) or less.

When the above-mentioned heat-exchange-type reaction vessel has a plurality of reaction tubes, it is preferable that U satisfy the above-mentioned specification in 50% or more of the total number of the above-mentioned plurality of reaction tubes, it is more preferable that U satisfy the above-mentioned specification in 80% or more of the total number of the above-mentioned plurality of reaction tubes, and it is further preferable that U satisfy the above-mentioned specification in 90% or more of the total number of the above-mentioned plurality of reaction tubes.

Examples of methods of adjusting U include changing of the circulation conditions for a heat medium, changing of the flow rate of nitrogen gas, changing of the proportion and the shape of the catalyst in the first catalyst layer, changing of the material and the shape of the diluent material in the first catalyst layer, and changing of the material, the diameter, or the thickness of the reaction tube.

F in Formula (*) is the supply amount (mol/h) of the above-mentioned raw material to the above-mentioned reaction tube. When the heat-exchange-type reaction vessel has a plurality of reaction tubes, F is the supply amount (mol/h) of the above-mentioned raw material per one tube of the above-mentioned plurality of reaction tubes. F is not particularly limited as long as ξ satisfies Expression (1), but from the viewpoint of maintaining the productivity of one or both of (meth)acrolein and (meth)acrylic acid, F is preferably 1 (mol/h) or more, more preferably 2.5 (mol/h) or more, and further preferably 6.5 (mol/h) or more. In addition, from the viewpoint of reducing the amount of heat generated in the catalyst layer, F is preferably 20 (mol/h) or less, more preferably 15 (mol/h) or less, and further preferably 10.5 (mol/h) or less.

When the above-mentioned heat-exchange-type reaction vessel has a plurality of reaction tubes, it is preferable that F satisfy the above-mentioned specification in 50% or more of the total number of the above-mentioned plurality of reaction tubes, it is more preferable that F satisfy the above-mentioned specification in 80% or more of the total number of the above-mentioned plurality of reaction tubes, and it is further preferable that F satisfy the above-mentioned specification in 90% or more of the total number of the above-mentioned plurality of reaction tubes.

A in Formula (*) is the inner surface area (m²) of the above-mentioned reaction tube with which the first catalyst layer from the raw material inlet side of the reaction tube comes into contact. A is not particularly limited as long as ξ satisfies Expression (1), but from the viewpoint of reducing the amount of heat generated in the catalyst layer, A is preferably 0.03 (m²) or more, more preferably 0.06 (m²) or more, and further preferably 0.09 (m²) or more. In addition, from the economical viewpoint of preventing the reaction vessel from becoming expensive, A is preferably 0.6 (m²) or less, more preferably 0.4 (m²) or less, and further preferably 0.25 (m²) or less.

When the above-mentioned heat-exchange-type reaction vessel has a plurality of reaction tubes, it is preferable that A satisfy the above-mentioned specification in 50% or more of the total number of the above-mentioned plurality of reaction tubes, it is more preferable that A satisfy the above-mentioned specification in 80% or more of the total number of the above-mentioned plurality of reaction tubes, and it is further preferable that A satisfy the above-mentioned specification in 90% or more of the total number of the above-mentioned plurality of reaction tubes.

In the production method of the present invention, the above-mentioned raw material can be supplied to the reaction tube as a raw material gas containing the raw material. The raw material concentration in the above-mentioned raw material gas is preferably 1% to 20% by volume, and more preferably 3% to 10% by volume. As described above, when the present invention is a production method of (meth)acrolein and (meth)acrylic acid, the raw material is at least one selected from propylene, isobutylene, tert-butanol, and methyl tert-butyl ether; and when the present invention is a production method of (meth)acrylic acid, the raw material is (meth)acrolein.

The above-mentioned raw material gas preferably contains 5% to 15% by volume of oxygen. As an oxygen source, air is preferable from the economical viewpoint. In addition, a gas, which is enriched with oxygen by adding pure oxygen to air, or the like may be used as necessary. In addition, the raw material gas preferably contains 5% to 50% by volume of water vapor.

The above-mentioned raw material gas may be one in which the above-mentioned raw material, oxygen, and water vapor have been diluted with an inert gas such as nitrogen gas and carbon dioxide gas. In addition, the above-mentioned raw material gas may contain a small amount of impurities such as lower saturated aldehydes, and the amount thereof is preferably as small as possible.

The space velocity of the above-mentioned raw material in the above-mentioned catalyst layer is preferably 200 to 5,000 h⁻¹.

The reaction pressure in the oxidation reaction of the above-mentioned raw material is preferably atmospheric pressure to a few atm. In addition, the temperature of the heat medium circulating the outer side of the above-mentioned reaction tube is preferably 230° C. to 450° C. The lower limit of the temperature of the above-mentioned heat medium is more preferably 250° C. or higher, and the upper limit thereof is more preferably 400° C. or lower.

As described above, according to the method of the present embodiment, the selectivity of (meth)acrylic acid can be improved in the synthesis of (meth)acrolein and/or (meth)acrylic acid.

### EXAMPLES

Hereinafter, the present invention will be specifically described with reference to examples. However, the present invention is not limited thereto.

In the examples and the comparative examples to be described later, and i are each as follows.

ξ=F×(m1/Σⱼ₌₁ⁱmj)/(U×A) (*)

In Formula (*), m1 is the catalyst charging amount (kg) in the first catalyst layer from the raw material inlet side of the reaction tube; mj is the catalyst charging amount (kg) in the j-th catalyst layer from the raw material inlet side of the reaction tube; j is an integer of 1 or more and i or less; F is the supply amount (mol/h) of the raw material supplied to the reaction tube; A is the inner surface area (m²) of the reaction tube with which the first catalyst layer from the raw material inlet side of the reaction tube comes into contact; and U is the overall heat transfer coefficient (W/m²/K) based on the inner surface area of the portion in the reaction tube with which both the first catalyst layer and the heat medium come into contact.

$$τ = m 1 / {\sum }_{k = 1}^{i} mk \qquad \text{(*} \text{*)}$$

In Formula (**), m1 is the catalyst charging amount (kg) in the first catalyst layer from the raw material inlet side of the reaction tube; mk is the catalyst charging amount (kg) in the k-th catalyst layer from the raw material inlet side of the reaction tube; and k is an integer of 1 or more and i or less.

(Compositional Ratio of Catalyst)

The atomic ratio of each of the elements was obtained by analyzing a component in which the catalyst had been dissolved in ammonia water by high-frequency inductively coupled plasma (ICP) optical emission spectrometry.

Apparatus used: inductively coupled plasma (ICP) optical emission spectrometer (Optima 8300 ICP-OES Spectrometer manufactured by PerkinElmer, Inc.)

(Calculation of Overall Heat Transfer Coefficient U)

The overall heat transfer coefficient U was calculated from the results of measuring the temperature distribution in the minute region of the first catalyst layer by supplying an inert gas composed of 21% by volume of oxygen and 79% by volume of nitrogen to the reaction tube at a temperature 50° C. lower than that of the heat medium circulating the outer side of the reaction tube.

(Analysis of Raw Material and Product)

The raw material and the product were analyzed by gas chromatography (device: GC-2014 manufactured by Shimadzu Corporation; column: DB-FFAP manufactured by J&W, 30 m×0.32 mm, film thickness 1.0 μm).

In addition, the reaction rate of methacrolein, the selectivity of generated methacrylic acid, and the yield of methacrylic acid were each defined as below.

Methacrolein reaction rate (%)=(β/α)×100

Methacrylic acid selectivity (%)=(γ/β)×100

Methacrylic acid yield (%)=(γ/α)×100
α is the substance amount (mol) of supplied methacrolein, β is the substance amount (mol) of reacted methacrolein, and γ is the substance amount (mol) of generated methacrylic acid.

(Evaluation of Amount of Heat Generated in Catalyst Layer)

A difference (ΔTmax) between the temperature of the portion showing the highest temperature in the catalyst layer and the temperature of the heat medium circulating the outer side of the reaction tube was used as an index for evaluating the amount of heat generated in the catalyst layer. ΔTmax was measured as follows. The temperature of the catalyst layer was measured by a thermocouple inserted into a protective tube installed in the center of the cross-section perpendicular to the longitudinal direction of the reaction tube. The protective tube is isolated from the reaction system, and the temperature measurement position can be changed by adjusting the length of the inserted thermocouple. The difference between the temperature of the catalyst layer measured this time and the temperature of the heat medium was defined as ΔT to calculate ΔT distribution. The maximum ΔT in the obtained ΔT distribution was defined as ΔTmax.

### Example 1

Using the multi-tubular heat-exchange-type reaction vessel having a heat medium bath shown in FIG. 1, methacrylic acid was produced by the oxidation reaction of methacrolein as follows. The above-mentioned reaction vessel had reaction tubes made of SUS304 and having an inner diameter of 27.2 mm and a length of 6 m at the inner part.

In each of the reaction tubes, two catalyst layers were formed using a cylindrical column-shaped catalyst in which the compositional ratio excluding oxygen was P_{1.1}Mo₁₂V_{0.6}Cu_{0.1}Fe_{0.05}Cs_{1.3} and which had a diameter of 6 mm and a height of 5 mm. 1,000 g of the catalyst and 250 g of alumina spheres having a diameter of 5 mm as a diluent material were mixed and charged to the first catalyst layer from the raw material inlet side of the reaction tube. In addition, 2,500 g of the catalyst was charged to the second catalyst layer. Table 1 shows the length of each of the catalyst layers, and the value of τ. An inert substance layer formed from alumina spheres having a diameter of 5 mm was formed between the end part on the raw material inlet side of the reaction tube and the catalyst layer.

Subsequently, while allowing the heat medium bath provided on the outer side of the reaction tube to circulate a heat medium, a raw material gas composed of 6.0% by volume of methacrolein, 10% by volume of oxygen, 10% by volume of water vapor, and 74.0% by volume of nitrogen was supplied to the reaction tube at a space velocity of 1,700 h⁻¹ to cause the oxidation reaction. As the heat medium, a salt melt composed of 50% by mass of potassium nitrate and 50% by mass of sodium nitrite was used, and the heat medium temperature was set to 310° C. Table 1 shows the supply amount F of methacrolein, the overall heat transfer coefficient U, the value of ξ, and the reaction rate of methacrolein at this time.

Thereafter, a continuous operation was carried out for 40 days while maintaining the reaction rate by adjusting the temperature of the heat medium. During the continuous operation, the temperature of the catalyst layer was periodically measured to calculate ΔT distribution. ΔTmax during the continuous operation period was 31° C. in the first catalyst layer and was 22° C. in the second catalyst layer. In addition, the average selectivity of methacrylic acid during the continuous operation period was 82%.

### Comparative Example 1

Using the same multi-tubular heat-exchange-type reaction vessel as in Example 1, methacrylic acid was produced by the oxidation reaction of methacrolein as follows.

Using the same catalyst as in Example 1 for each of the reaction tubes to form one layer of a catalyst layer. Only 3,500 g of the catalyst was charged to the catalyst layer. Table 1 shows the length of the catalyst layer, and the value of τ. The same inert substance layer as in Example 1 was formed between the end part on the raw material inlet side of the reaction tube and the catalyst layer.

Subsequently, while circulating the heat medium by the same method as in Example 1, the raw material gas was supplied by the same method as in Example 1 to cause the oxidation reaction. Table 1 shows the supply amount F of methacrolein, the overall heat transfer coefficient U, the value of ξ, and the reaction rate of methacrolein.

Thereafter, a continuous operation was carried out while maintaining the reaction rate by adjusting the temperature of the heat medium. However, two days after the start of the operation, ΔTmax of the catalyst layer reached 200° C., and the operation had to be stopped. In addition, the methacrylic acid selectivity before stopping the operation was 77%.

### Examples 2 to 4 and Comparative Examples 2 and 3

A simulation reproducing the reaction results of Example 1 was produced as follows.

The same conditions as in Example 1 were used for forming a catalyst layer and supplying a reaction gas to calculate the reaction rate with respect to the temperature and the concentration of each of minute regions divided in the longitudinal direction of the reaction tube from the inlet of the reaction tube. From the obtained reaction rate, a material balance equation and a heat balance equation were created in the longitudinal direction of the reaction tube to calculate the temperature in a region adjacent to the product outlet side and the concentration of each compound present in the reaction tube. This was repeated upto a region on the most product outlet side, and fitting of reaction rate parameters was performed so that the same ΔTmax, the same methacrolein reaction rate, and the same methacrylic acid selectivity as in Example 1 were obtained. The change in the substance amount in the longitudinal direction of the reaction tube in the minute region was obtained by obtaining the sum of M1 (mol/s) of all reactions occurring in the reaction tube when the product of the stoichiometric coefficient of the compound based on the reaction formula of each of the reactions occurring in the reaction tube, the reaction rate per volume (mol/m³/s), and the volume (m³) of the minute region was defined as M1. In addition, a temperature change dT1′ (K) in the minute region in the longitudinal direction of the reaction tube was obtained by Formula (***)′ when a difference between the temperature of the heat medium circulating the outer side of the reaction tube and the average temperature in the minute region was defined as dT2′ (K), and when the product of the reaction rate per volume (mol/m³/s), the reaction heat amount (J/mol), and the volume (m³) of the minute region was defined as M2 (W).

dT1′=[U×inner surface area(m²) of reaction tube with which minute region is in contact with×dT2′+sum of M2 of all reactions occurring in reaction tube]/[mass flow rate(g/s) of raw material gas×specific heat(J/g/K) of raw material gas] (***)′

It was confirmed that the same ΔTmax, the same methacrolein reaction rate, and the same methacrylic acid selectivity as in Example 1 were obtained when the heat medium circulation conditions were set to be the same as those in Example 1 by using the produced simulation.

Subsequently, the simulation produced above was carried out. Table 1 shows the length of the catalyst layer, the catalyst charging amount, the value of τ, the value of U, and the value of ξ in each of the examples and comparative examples. The circulation conditions for the heat medium and the supply conditions for the raw material gas were the same as those in Example 1. Table 1 shows ΔTmax, the methacrolein reaction rate, and the methacrylic acid selectivity which were obtained by the simulation.

TABLE 1

| | | Example | Comparative | Example | Example | Example | Comparative | Comparative |
|---|---|---|---|---|---|---|---|---|
| | | 1 | Example 1 | 2 | 3 | 4 | Example 2 | Example 3 |
| Number i of | | 2 | 1 | 2 | 2 | 2 | 2 | 2 |
| catalyst layer | | | | | | | | |
| Length | First | 1700 | 5000 | 1360 | 2550 | 1700 | 1700 | 1430 |
| of | layer | | | | | | | |
| catalyst | Second | 3500 | — | 3780 | 2800 | 3500 | 3500 | 3500 |
| layer | layer | | | | | | | |
| [mm] | Total | 5200 | 5000 | 5140 | 5350 | 5200 | 5200 | 4930 |
| Inner surface area | | 0.145 | 0.427 | 0.116 | 0.218 | 0.145 | 0.145 | 0.122 |
| A of first layer | | | | | | | | |
| [m²] | | | | | | | | |
| Catalyst | First | 1000 | 3500 | 500 | 1500 | 1000 | 1000 | 1000 |
| amount | layer | | | | | | | |
| [g] | Second | 2500 | 0 | 3000 | 2000 | 2500 | 2500 | 2500 |
| | layer | | | | | | | |
| | Total | 3500 | 3500 | 3500 | 3500 | 3500 | 3500 | 3500 |
| τ | | 0.29 | 1.00 | 0.14 | 0.43 | 0.29 | 0.29 | 0.29 |
| Supply amount F | | 8.3 | 8.3 | 8.3 | 8.3 | 8.3 | 8.3 | 8.3 |
| of methacrolein | | | | | | | | |
| [mol/hr] | | | | | | | | |
| Overall heat | | 70 | 70 | 70 | 70 | 63 | 56 | 70 |
| transfer | | | | | | | | |
| coefficient U | | | | | | | | |
| [W/m²/K] | | | | | | | | |
| ζ | | 0.233 | 0.278 | 0.146 | 0.233 | 0.259 | 0.292 | 0.277 |
| [mol · K/h/W] | | | | | | | | |
| Methacrolein | | 74 | 74 | 74 | 74 | 75 | 75 | 74 |
| reaction rate [%] | | | | | | | | |
| ΔTmax | First | 31 | 200 | 29 | 31 | 36 | 42 | 40 |
| of | layer | | | | | | | |
| catalyst | Second | 22 | — | 33 | 25 | 35 | 41 | 39 |
| layer | layer | | | | | | | |
| [° C.] | | | | | | | | |

| | | | | | | | |
|---|---|---|---|---|---|---|---|
| Continuous | 40 | 2 | — | — | — | — | — |
| operation period | | | | | | | |
| [day] | | | | | | | |
| Methacrylic acid | 82 | 77 | 82 | 82 | 81 | 79 | 81 |
| selectivity [%] | | | | | | | |

As shown in Table 1, in Example 1 in which the oxidation reaction of methacrolein was caused under the condition satisfying Expression (1), the continuous operation for 40 days could be carried out with a higher methacrylic acid selectivity than in the comparative examples. In addition, Examples 2 to 4 also showed stable ΔTmax and favorable methacrylic acid selectivity.

On the other hand, in Comparative Example 1, since the amount of heat generated in the catalyst layer increased rapidly, the operation was stopped in 2 days, which resulted in a low methacrylic acid selectivity. In addition, in Comparative Examples 2 to 3, ΔTmax was high as compared to the examples, and it was shown that the amount of heat generated in the first catalyst layer was large for the heat removal capacity of the reaction vessel.

### INDUSTRIAL APPLICABILITY

The present invention is industrially useful since one or both of (meth)acrolein and (meth)acrylic acid can be produced with a high selectivity by preventing the runaway reaction in the heat-exchange-type reaction vessel and preventing the excessive progression of a reaction and a local catalyst degradation.

### REFERENCE SIGNS LIST

1: Reaction vessel
2: Reaction tube
3: Baffle plate
4: Raw material inlet part
5: Product outlet part
6: Heat medium inlet part
7: Heat medium outlet part
8: Heat medium bath
9: First partition plate
10: Second partition plate

What is claimed is:

1. A production method of one or both of (meth)acrolein and (meth)acrylic acid using a heat-exchange-type reaction vessel comprising a reaction tube at an inner part, the production method comprising
causing an oxidation reaction of a raw material supplied to the reaction tube while circulating a heat medium to an outer side of the reaction tube to produce one or both of (meth)acrolein and (meth)acrylic acid,
wherein the reaction tube comprises i layers, which are a plurality of catalyst layers having different catalyst charging amounts per unit volume, in a longitudinal direction of the reaction tube, provided that i is an integer of 2 or more, and
the oxidation reaction satisfies Expression (1),
ξ≤0.275(mol·K/h/W) (1)
provided that the following formula is satisfied,
ξ=F×(m1/Σⱼ₌₁ⁱmj)/(U×A) (*)
in Formula (*), m1 is a catalyst charging amount (kg) in a first catalyst layer from a raw material inlet side of the reaction tube; mj is a catalyst charging amount (kg) in a j-th catalyst layer from the raw material inlet side of the reaction tube; j is an integer of 1 or more and i or less; F is a supply amount (mol/h) of the raw material supplied to the reaction tube; A is an inner surface area (m²) of the reaction tube with which the first catalyst layer from the raw material inlet side of the reaction tube comes into contact; and U is an overall heat transfer coefficient (W/m²/K) based on an inner surface area of a portion in the reaction tube with which both the first catalyst layer and the heat medium come into contact.

2. The production method according to claim 1, wherein the oxidation reaction further satisfies Expression (1′),
0.002≤ξ≤0.275(mol·K/h/W) (1′).

3. The production method according to claim 1, wherein the oxidation reaction further satisfies Expression (1″),
ξ≤0.24(mol·K/h/W) (1″).

4. The production method according to claim 1, wherein the oxidation reaction further satisfies Expression (2),
0.25≤τ≤0.5 (2)
provided that the following formula is satisfied,
$$τ = m 1 / {\sum }_{k = 1}^{i} mk \qquad \text{(*} \text{*)}$$
in Formula (**), m1 is the catalyst charging amount (kg) in the first catalyst layer from the raw material inlet side of the reaction tube; mk is a catalyst charging amount (kg) in a k-th catalyst layer from the raw material inlet side of the reaction tube; and k is an integer of 1 or more and i or less.

5. The production method according to claim 4, wherein the oxidation reaction further satisfies Expression (2′),
0.26≤τ≤0.5 (2′).

6. The production method according to claim 1, wherein i as the number of the catalyst layers of the reaction tube is 2 to 4.

7. The production method according to claim 1, wherein U is 40 to 400 (W/m²/K).

8. The production method according to claim 1, wherein U is 50 to 300 (W/m²/K).

9. The production method according to claim 1, wherein F is 1 to 20 (mol/h).

10. The production method according to claim 1, wherein F is 2.5 to 15 (mol·h⁻¹).

11. The production method according to claim 1, wherein A is 0.03 to 0.6 (m²).

12. The production method according to claim 1,
wherein the raw material is at least one selected from propylene, isobutylene, tert-butanol, and methyl tert-butyl ether, and
one or both of the (meth)acrolein and the (meth)acrylic acid are (meth)acrolein and (meth)acrylic acid.

13. The production method according to claim 1,
wherein the raw material is (meth)acrolein, and
one or both of the (meth)acrolein and the (meth)acrylic acid are (meth)acrylic acid.

14. The production method according to claim 12,
wherein a catalyst having a formulation represented by Formula (I) is used in the catalyst layer,
Moₐ₁Bi_{b1}Fe_{c1}M_{d1}Xₑ₁Y_{f1}Z_{g1}Siₕ₁Oᵢ₁ (I)
in Formula (I), Mo, Bi, Fe, Si, and O each represent molybdenum, bismuth, iron, silicon, and oxygen; M represents at least one element selected from the group consisting of cobalt and nickel; X represents at least one element selected from the group consisting of chromium, lead, manganese, calcium, magnesium, niobium, silver, barium, tin, tantalum, and zinc; Y represents at least one element selected from the group consisting of phosphorus, boron, sulfur, selenium, tellurium, cerium, tungsten, antimony, and titanium; Z represents at least one element selected from the group consisting of lithium, sodium, potassium, rubidium, cesium, and thallium; and a1, b1, c1, d1, e1, f1, g1, h1, and i1 represent an atomic ratio of each of the elements, provided that when a1=12, b1=0.01 to 3, c1=0.01 to 5, d1=1 to 12, e1=0 to 8, f1=0 to 5, g1=0.001 to 2, h1=0 to 20, and i1 is an atomic ratio of oxygen required to satisfy a valence of the element.

15. The production method according to claim 13,
wherein a catalyst having a formulation represented by Formula (II) is used in the catalyst layer,
Pₐ₂Mo_{b2}V_{c2}Cu_{d2}Xₑ₂Y_{f2}Z_{g2}Oₕ₂ (II)
in Formula (II), P, Mo, V, Cu, and O each represent phosphorus, molybdenum, vanadium, copper, and oxygen; X represents at least one element selected from the group consisting of antimony, bismuth, arsenic, germanium, zirconium, tellurium, silver, selenium, silicon, tungsten, and boron; Y represents at least one element selected from the group consisting of iron, zinc, chromium, magnesium, tantalum, cobalt, manganese, barium, gallium, cerium, and lanthanum; Z represents at least one element selected from the group consisting of potassium, rubidium, cesium, and thallium; and a2, b2, c2, d2, e2, f2, g2, and h2 represent an atomic ratio of each of the elements, provided that when b2=12, a2=0.5 to 3, c2=0.01 to 3, d2=0.01 to 2, e2=0 to 3, f2=0 to 3, g2=0.01 to 3, and h2 is an atomic ratio of oxygen required to satisfy a valence of the element.

16. The production method according to claim 2,
wherein the raw material is at least one selected from propylene, isobutylene, tert-butanol, and methyl tert-butyl ether, and
one or both of the (meth)acrolein and the (meth)acrylic acid are (meth)acrolein and (meth)acrylic acid.

17. The production method according to claim 2,
wherein the raw material is (meth)acrolein, and
one or both of the (meth)acrolein and the (meth)acrylic acid are (meth)acrylic acid.

18. The production method according to claim 16,
wherein a catalyst having a formulation represented by Formula (I) is used in the catalyst layer,
Moₐ₁Bi_{b1}Fe_{c1}M_{d1}Xₑ₁Y_{f1}Z_{g1}Siₕ₁Oᵢ₁ (I)
in Formula (I), Mo, Bi, Fe, Si, and O each represent molybdenum, bismuth, iron, silicon, and oxygen; M represents at least one element selected from the group consisting of cobalt and nickel; X represents at least one element selected from the group consisting of chromium, lead, manganese, calcium, magnesium, niobium, silver, barium, tin, tantalum, and zinc; Y represents at least one element selected from the group consisting of phosphorus, boron, sulfur, selenium, tellurium, cerium, tungsten, antimony, and titanium; Z represents at least one element selected from the group consisting of lithium, sodium, potassium, rubidium, cesium, and thallium; and a1, b1, c1, d1, e1, f1, g1, h1, and i1 represent an atomic ratio of each of the elements, provided that when a1=12, b1=0.01 to 3, c1=0.01 to 5, d1=1 to 12, e1=0 to 8, f1=0 to 5, g1=0.001 to 2, h1=0 to 20, and i1 is an atomic ratio of oxygen required to satisfy a valence of the element.

19. The production method according to claim 17,
wherein a catalyst having a formulation represented by Formula (II) is used in the catalyst layer,
Pₐ₂Mo_{b2}V_{c2}Cu_{d2}Xₑ₂Y_{f2}Z_{g2}Oₕ₂ (II)
in Formula (II), P, Mo, V, Cu, and O each represent phosphorus, molybdenum, vanadium, copper, and oxygen; X represents at least one element selected from the group consisting of antimony, bismuth, arsenic, germanium, zirconium, tellurium, silver, selenium, silicon, tungsten, and boron; Y represents at least one element selected from the group consisting of iron, zinc, chromium, magnesium, tantalum, cobalt, manganese, barium, gallium, cerium, and lanthanum; Z represents at least one element selected from the group consisting of potassium, rubidium, cesium, and thallium; and a2, b2, c2, d2, e2, f2, g2, and h2 represent an atomic ratio of each of the elements, provided that when b2=12, a2=0.5 to 3, c2=0.01 to 3, d2=0.01 to 2, e2=0 to 3, f2=0 to 3, g2=0.01 to 3, and h2 is an atomic ratio of oxygen required to satisfy a valence of the element.
